(19) United States
(12) Patent Application Publication
(10) Pub. No.: US 2023/0381206 A1
(43) Pub. Date: Nov. 30, 2023

(54) DRINK CONTAINING A FROZEN CANNABIS PRODUCT AND METHOD OF MAKING

(71) Applicant: Hot Ice, LLC, Livonia, MI (US)

(72) Inventor: Matthew Demorest, San Juan, AR (US)

(21) Appl. No.: 18/201,045

(22) Filed: May 23, 2023

Related U.S. Application Data
(60) Provisional application No. 63/345,268, filed on May 24, 2022.

(51) Int. Cl. A61K 31/00; A61K 9/00; A23L 33/105; A23L 2/52

(52) U.S. Cl. CPC A61K 31/658; A61K 9/0095; A23L 33/105; A23L 2/52

(57) ABSTRACT

The frozen cannabis product may be included in a drink that also includes an ingestible liquid, such as soda, water, or juice. The frozen cannabis product is formed from a base constituent and at least one of tetrahydrocannabinol (THC) and cannabidiol (CBD) that are admixed together in liquid form and then frozen to make cannabis ice. The cannabis ice melts with a known amount of the THC and/or CBD, thereby allowing a user to refill their drink as many times as they want while using the same cannabis ice with minimal risk of an overdosing on the THC and/or CBD.

### CROSS-REFERENCE TO RELATED APPLICATION

This application claims priority to U.S. Provisional Patent Application No. 63/345,268, filed on May 24, 2022, and entitled “DRINK CONTAINING A FROZEN CANNABIS PRODUCT AND METHOD OF MAKING,” the entire contents of which is herein incorporated by reference.

### BACKGROUND

### 1. Field

The present disclosure is related generally to cannabis products for human ingestion.

### 2. Related Art

Cannabis concentrates can contain very high levels of tetrahydrocannabinol (THC), cannabidiol (CBD), or a range of other cannabinoids. These concentrates may be ingested by users through a range of different means beyond smoking, including through food products (such as gummies) or from a vaporizer that vaporizes the concentrates through a process called dabbing. There is a continuing demand for new and novel cannabis products.

### SUMMARY

One aspect of the present disclosure is related to a drink that includes an ingestible liquid with a frozen cannabis product provided therein. The frozen cannabis product is formed from a base constituent and at least one of tetrahydrocannabinol (THC) and cannabidiol (CBD) admixed together.

Implementations may include any one or combination of the following features. The base constituent may be water. The frozen cannabis product may have a cube shape. The frozen cannabis product may have a spherical shape. The at least one of THC and CBD may be non-uniformly distributed across the frozen cannabis product. A central area of the frozen cannabis product may have a higher concentration of the at least one of THC and CBD than an outer area of the frozen cannabis product. The frozen cannabis product may include ten to forty-five milligrams (10-45 mg) of CBD. The frozen cannabis product may include one to fifty milligrams (1-50 mg) of THC.

Another aspect of the present disclosure is related to a frozen cannabis product that includes a base constituent admixed with at least one of tetrahydrocannabinol (THC) and cannabidiol (CBD) and is frozen.

Implementations may include any one or combination of the following features. The base constituent may be water. The frozen cannabis product may have a cube shape. The frozen cannabis product may have a spherical shape. The at least one of THC and CBD may be non-uniformly distributed across the frozen cannabis product. A central area of the frozen cannabis product may have a higher concentration of the at least one of THC and CBD than an outer area of the frozen cannabis product. The frozen cannabis product may include ten to forty-five milligrams (10-45 mg) of CBD. The frozen cannabis product may include one to fifty milligrams (1-50 mg) of THC.

Another aspect of the present disclosure is related to a method of making a frozen cannabis product. The method includes the step of admixing a base liquid with at least one of tetrahydrocannabinol (THC) and cannabidiol (CBD) to form a cannabis mixture. The method also includes the step of freezing the cannabis mixture to form the frozen cannabis product.

Implementations may include one or any combination of the following features. The frozen cannabis product may include ten to forty-five milligrams (10-45 mg) of CBD. The frozen cannabis product may include one to fifty milligrams (1-50 mg) of THC. The method may further include the step of inserting the frozen cannabis product into an ingestible liquid to form a drink. The base liquid may be water. The frozen cannabis product may have a spherical shape or a cube shape.

### DETAILED DESCRIPTION OF THE DRAWINGS

These and other features and advantages of the invention will become more readily appreciated when considered in connection with the following description of the presently preferred embodiments, appended claims and accompanying drawings, in which:

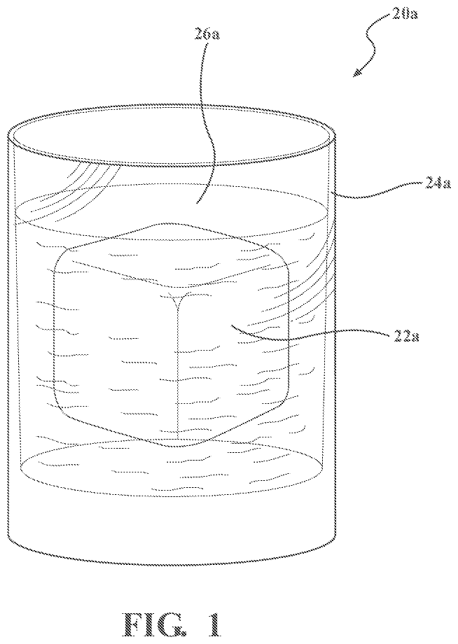

FIG. 1 is a perspective elevation view of a first embodiment of the present disclosure illustrating a frozen cannabis product having the shape of a cube in a rocks glass;

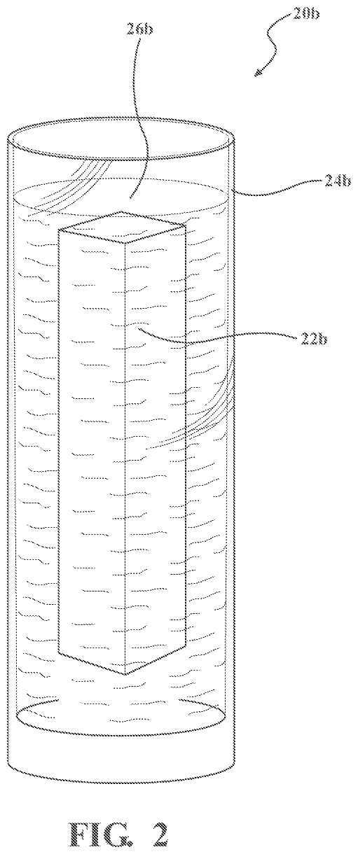

FIG. 2 is a perspective view of a second embodiment of the present disclosure illustrating a frozen cannabis product having the shape of a cuboid in a Collins glass;

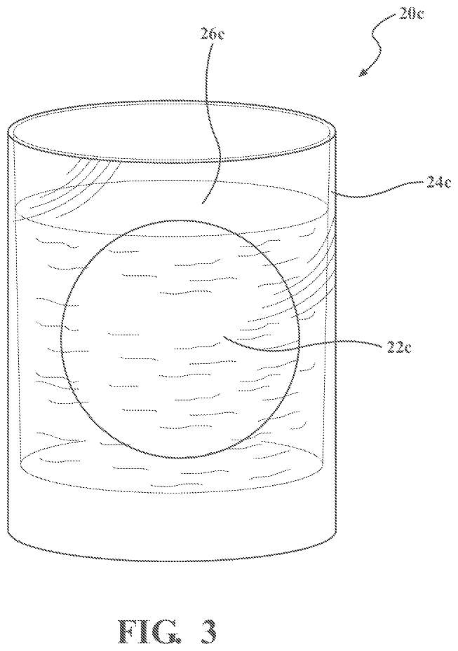

FIG. 3 is a perspective view of a third embodiment of the present disclosure illustrating a frozen cannabis product having the shape of a sphere in a rocks glass;

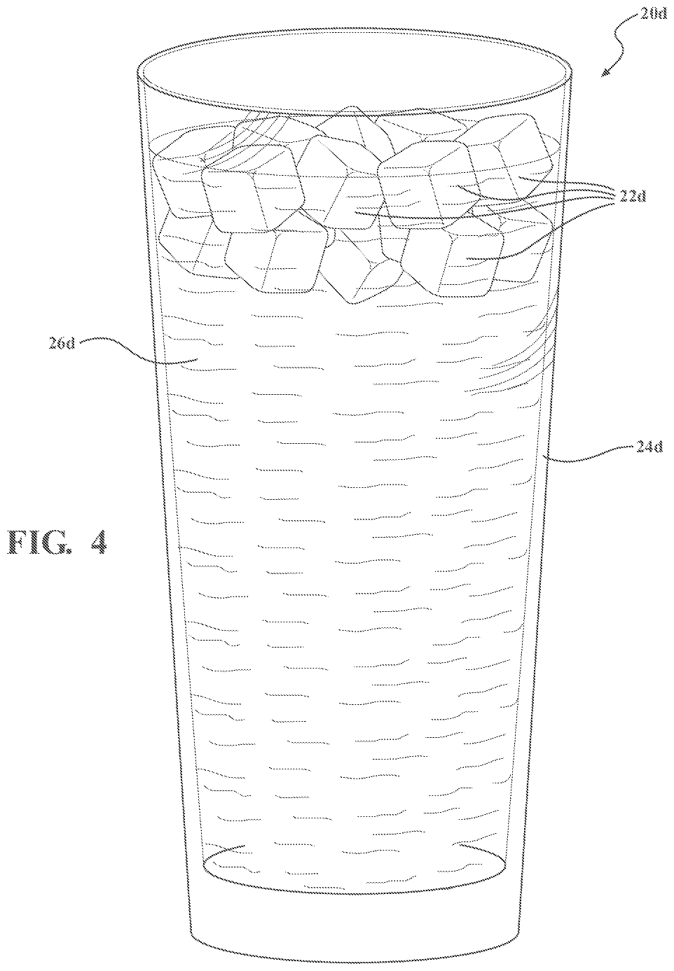

FIG. 4 is a perspective view of a fourth embodiment of the present disclosure illustrating a frozen cannabis product having the shape of a cube in a rocks glass;

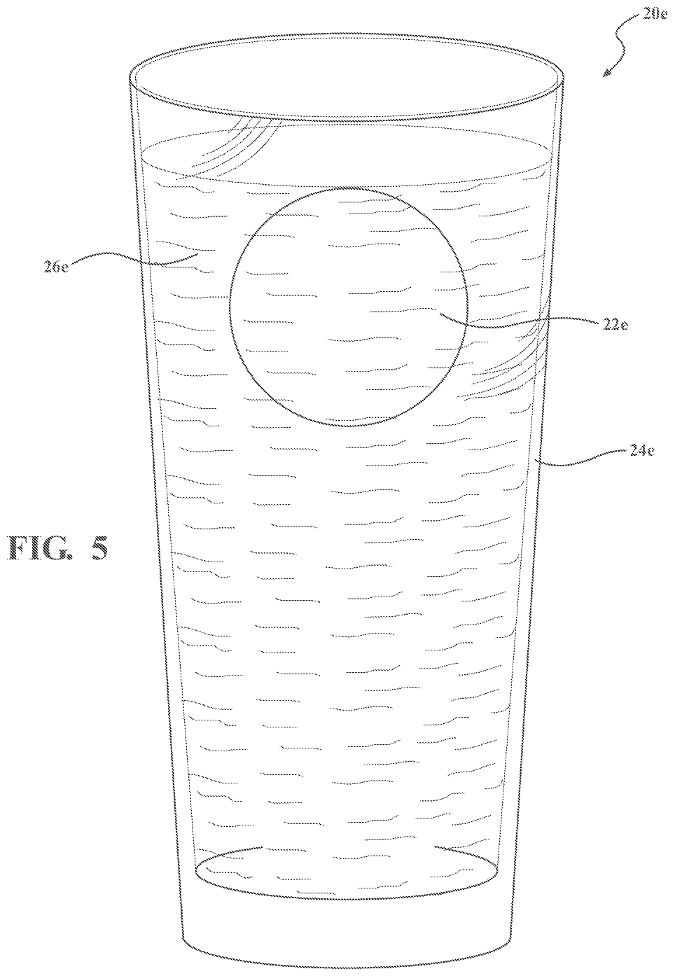

FIG. 5 is a perspective view of a fifth embodiment of the present disclosure illustrating a frozen cannabis product having the shape of a sphere in a rocks glass;

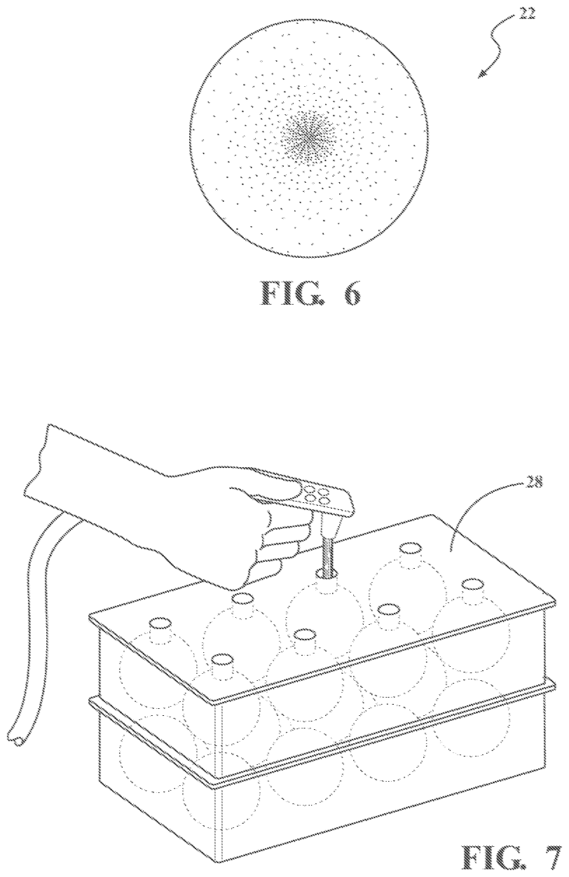

FIG. 6 is a cross-sectional view of an example cannabis ice and illustrating a concentration of a cannabis product within different locations of the cannabis ice; and

FIG. 7 is a perspective view of a cannabis mixture in a tray prior to freezing.

### DETAILED DESCRIPTION OF THE ENABLING EMBODIMENTS

Referring to the drawing, an exemplary embodiment of a drink 20 containing a frozen cannabis product is provided. The frozen cannabis product (hereinafter referred to as a “cannabis ice product 22” has the form of a piece of ice that has a measured amount of a cannabis concentrate or product contained within it. As discussed in further detail below, the cannabis ice product 22 can be inserted into a liquid, such as water or a soda, and will slowly melt, thereby releasing the cannabis product into the fluid in a slow and controlled manner for ingestion by a user as the user drinks the liquid. The cannabis ice product 22 can have a range of different shapes and sizes.

For example, in the embodiments of FIGS. 1 and 4, the cannabis ice product 22a, has the shape of a cube, and the cannabis ice product 22a has a larger size in FIG. 1 than in FIG. 4, which contains multiple smaller cannabis ice product 22 cubes. In the embodiment of FIG. 2, the cannabis ice product 22b has the shape of a cuboid, and in the embodiments of FIGS. 3 and 5, the cannabis ice product 22c, 22e has the shape of a sphere. The shape of the cannabis ice product 22 influences its volume to surface area ratio, which will contribute to how quickly the cannabis ice product 22 melts in the drink 20.

In the exemplary embodiments, the drink 20 is provided in a glass 24, though any suitable type of vessel may be provided. The glass 24 may be, for example, a rocks glass 24a (FIGS. 1 and 3), a Collins glass 24b (FIG. 2), or a pint glass 24c, 24e (FIGS. 4 and 5). The liquid 26 may be any suitable liquid for human consumption, such as soda, water, juice, a seltzer, etc. The drink 20 may include only a single cannabis ice product 20 or multiple cannabis ice products 20 (for example, see FIG. 4).

The volume to surface area ratio of the cannabis ice product 22, the size and shape of the glass 24, and the amount of liquid 26 in the glass 24 will all contribute to the rate that the cannabis ice product 22 will melt within the liquid 26. In the embodiment of FIG. 1, the cannabis ice product 22a has dimensions of approximately one inch by one inch by one inch (1″×1″×1″) and the glass 24a is a rocks glass with a relatively low volume. This combination gives the cannabis ice product 22a a large volume to surface area ratio within a relatively low volume of liquid 26a, thereby ensuring that the cannabis ice product 22a melts into the liquid 26a very slowly so that the user can enjoy their drink 20a for hours before the cannabis ice product 22a fully melts. The user can even refill the liquid 26a in their drink 20a multiple times while using the same cannabis ice product 22a and with knowledge of the total amount the cannabis product that will be ingested because the cannabis ice product 22a is the only source of the cannabis product in the drink 20a, no matter how many times the liquid 26a is refilled. In some embodiments, such as the embodiment of FIG. 4, multiple cannabis ice products 22d having different dimensions and/or shapes can be included in the drink 20d and in a larger volume of liquid 26d. Multiple, smaller cannabis ice products 22d in a larger volume of liquid 26d will melt comparatively more quickly so that the user can ingest the cannabis product contained in the cannabis ice products 22d more quickly. In other words, the user may select the appropriate combination of cannabis ice product 22 and glass 24 to achieve the desired effect.

In many preferred embodiments, each cannabis ice product 22 contains a single measured dose of a cannabis concentrate or product. In some embodiments, the cannabis product is decarboxylated cannabinoids, including Tetrahydrocannabinol (THC) and/or Cannabidiol (CBD). In some embodiments, other cannabis products may be provided in addition to or in substitute of THC and CBD. In some embodiments, the cannabis product includes a combination of a cannabinoid; a terpenes and sesquiterpenes; a flavonoid and phenol; a fatty acid; an amino acid; and additional additives.

The cannabinoids may be selected from a group consisting of: Cannabigerovarin (CBGV), Cannabigerovarinic acid (CBGVA), Cannabigerol (CBG), Cannabigerolic acid (CBGA), Cannabigerolic acid methylether, Cannabinerolic acid, Cannabinerol, Carmagerol, rac-6′-Epoxycannabigerol, rac-6′-Ep oxy cannabigerolic acid, rac-6′-Epoxycannabinerol, rac-6′-Epoxycannabinerolic acid, γ-Eudesmyl cannabigerolate, y-Cainyl cannabigerolate, Sesquicannabigerol, Deprenyl O-methyl cannabigerolic acid (Amorfrutin 2), 5-Acetyl-4-hydroxycannabigerol, Acetylcannabigeroquinol, Cannabigeroquinone, Abnormal cannabigerol, Acetyl abnormal hydrocannabigeroquinol, Abnormal cannabigeroquinol, 2′-Hydroxy-1′, 2′-dihydrocannabichromene (Cyclo-CBG), Cannabiorcichromene, Cannabiorcichromenic acid, Chlorcannabiorcichromenic acid, Cannabivarichromene (CB CV), Cannabichromevarinic acid, Cannabichromene (CBC), Cannabichromenic acid, 4-Acetoxycannabichromene, Anthopogochromenic acid, Confluentin, Daurichromenic acid, 8′-Hydroxyisocannabichromene, 4-Acetoxycannabichromene, Cannabidiorcol, Cannabidivarin (CBDV), Cannabidivarinic acid, nor-Cannabidiol, Cannabidiol (CBD), O-Methlycannabidiol, O-Propylcannabidiol, O-Pentylcannabidiol, Cannabidiolic acid (CBDA), CBDA-THC ester, Ferruginene C, Cannabioxepane, Cannabinodivarin, Cannabinodiol, Cannabifuran, Dehydrocannabifuran, trans-Δ-8-Tetrahydrocannabinol (Δ-8-THC), trans-Δ-8-Tetrahydrocannabinolic acid, 10α-Hydroxy trans-Δ-8-tetrahydrocannabinol, 10β-Hydroxy trans-Δ-8-tetrahydrocannabinol, 11-Acetoxy-Δ-8-tetrahydrocannabinoic acid, 10-Hydroxy-9-oxo-Δ-8-tetrahydrocannabinol, Δ-9-trans-Tetrahydrocannabiorcol, Δ-9-trans-Tetrahydrocannabiorcolic acid, Δ-9-trans-Tetrahydrocannabivarin (THCV), Δ-9-trans-Tetrahydrocannabivarinic acid, Δ-9-trans-nor-Tetrahydrocannabinol, Δ-9-trans-nor-Tetrahydrocannabinolic acid, Δ-9-trans-Tetrahydrocannabinol (→-9-THC), Δ-9-trans-Tetrahydrocannabinolic acid A, Δ-9-trans-Tetrahydrocannabinolic acid B, 8α-Hydroxy-Δ-9-trans-tetrahydrocannabinol, 8β-Hydroxy-Δ-9-trans-tetrahydrocannabinol, 8-Oxo-Δ-9-trans-tetrahydrocannabinol, O-Propyl-Δ-9-trans-tetrahydrocannabinol, O-Pentyl-Δ-9-trans-tetrahydrocannabinol, 2-Formyl-Δ-9-trans-tetrahydrocannabinol, P-Fenchyl Δ-9-trans-Tetrahydrocannabinolate, α-Fenchyl Δ-9-trans-Tetrahydrocannabinolate, Bornyl Δ-9-trans-Tetrahydrocannabinolate, epi-Bornyl Δ-9-trans-Tetrahydrocannabinolate, α-Terpinyl Δ-9-trans-Tetrahydrocannabinolate, 4-Terpinyl Δ-9-trans-Tetrahydrocannabinolate, γ-Eudesmyl Δ-9-trans-Tetrahydrocannabinolate, α-Cadinyl Δ-9-trans-Tetrahydrocannabinol ate, Hexahydrocannabinol, Hydroxy Δ-9,11-hexahydrocannabinol, Methylen-bis Δ-9-trans-Tetrahydrocannabinol (Cannabisol), Tetrahydrocannabinol epoxide, Δ-9-trans-Tetrahydrocannabinol glycol (cannabiripsol), 6a,7,10a-Trihydroxy-Δ-9-tetrahydrocannabinol, Δ-9-cis-Tetrahydrocannabivarin, Δ-9-cis-Tetrahydrocannabinol, Cannabicitran (citrilidene-cannabis)(CBT), Cannabiorcicitran, Bis-nor cannabitriol, Bis-nor-Cannabitriol isomer, 10-O-Ethyl bis-nor cannabitriol, Isocannabitriol, Cannabitriol, Cannabitriol isomer, 10-O-Ethyl cannabitriol isomer, 10-Oxo-46a(10a)-tetrahydrocannabinol, 9,10-Anhydrocannabitirol, Cannabiglendol, 7, 8-D ehydro-10-O-ethylcannabitriol, Δ-7-cis-Isotetrahydrocannabivarin, Δ-7-trans-Isotetrahydrocannabivarin, Δ-7-trans-isotetrahydrocannabinol, Bis-nor-cannabielsoin, Bis-nor-Cannabielsoic acid B, Cannabielsoin (CBE), Cannabielsoic acid A, Cannabielsoic acid B, Ferruginene A, Ferruginene B, Cannabiorcicyclol, Cannabiorcicyclolic acid, Cannabicyclovarin, Cannabicyclol (CBL), Cannabicyclolic acid, Anthopogocyclolic acid, Rhododaurichromanic acid A, Cannabiorcol, nor-Cannabivarin, Cannabivarin (CBV), nor-Cannabinol, Cannabinol (CBN), Cannabinolic acid, O-Methylcannabinol, O-Propylcannabinol, O-Penthylcannabinol, 7-Hydroxcannabinol, 8-Hydroxycannabinol, 8-Hydroxcannabinolic acid, 7,8-Dihydrocannabinol, 4-Terpenyl cannabinolate, Cannabicoumaronone, Cannabicourmarononic acid, Bisnor-Cannabichromanone, Cannabichromanone, (6aR)-Cannabichromanone B, (6aR)-Cannabichromanone C, Cannabichromanone, Cannabimovone, Anhydrocannabimovone, Dem ethyl decarb oxy am orfrutin A, Tetrahydrocannabiphorol (THCP), Cannabidiphorol (CBDP), and Cannabimovone (CBM).

The terpenes and sesquiterpenes may be selected from the group consisting of: Beta-Myrcene, Alpha-Pinene, Cis-Ocimene, Beta-Curcumene, Terpinolene, Camphene, Trans-Ocimene, Alpha-Terpinene, Piperitone, Eucalyptol, Linalool, Fenchyl Alcohol, Beta-Pinene, Citronellol, Borneol, Citronellal, D/L-Fenchone, Geraniol, D-Limonene, 3-Carene, Geranyl Acetate, Cuminaldehyde, Alpha-Phellandrene, Alpha-Thuj one, D/L-Menthol, Linalyl Acetate, Isopulegol, Carvone, Carvacrol, Gamma Terpinene, Menthofuran, Sabinene Hydrate, Nerol, Sabinene, Thymol, Camphor, Pulegone, Bornyl Acetate, Alpha-Terpineol, M-Cymene, Isocannabispiran, Beta-Farnesene, Isoborneol, Cis-Citral, Beta-Caryophyllene, Ledene, Alpha-Curcumene, Alpha-Bisabolol, Alpha-Humulene, Alpha-Gurjunene, Trans-Nerolidol, Caryophyllene Oxide, Beta Cedrene, Valencene, Alpha Cedrene, Thujopsene, Alpha-Farnesene, Cis-Nerolidol, Cedrol, Guaiazulene, Farnesol, Cuparene, Isolongifolene, Gauiol, and Phytol.

The flavonoids and phenols can be selected from the group consisting of: Phloroglucinol, Cannabistilbene, Cannabispiran, Cannflavin C, 2-Carene, Quercetin, Isocannabispiran, Alpha-Cannabi spiranol, Kaempferol, Dihydro-Resveratrol, Vincenin, Cytisoside, Chrysoeriol, Luteolin-Glucoside, Cannflavin A, Vitexin, Apigenin-Glucoside, Apigenin, Cannflavin B, Orientin, Luteolin-Glucoside, and Isovitexin.

The fatty acids can be selected from the group consisting of: Miristic acid, Pentadecanoic Acid, Palmitic acid, Palmitoleic acid, Eptadecanoic acid, Cis-10 cptadecanoic acid, Stearic acid, Elaidic acid, Oleic acid, Linoleic acid, Alpha-linoleic acid, Gamma-linoleic acid, Arachic aicd, Cis-11-eicosenoic acid, Linolenic acid, Heneicosanoic acid, Cis-11,14-eicosadienoic acid, Behenic acid, Cis-8,11,14-eicosatrienoic acid, Erucic acid, Cis-11,14,17-eicosatrienoic acid, Arachidonic acid, Cis-13,16-docosadienoic acid, Lignoceric acid, Cis-5, 8,11,14,17-eicosapentaenoic acid, and Nervonic acid.

The amino acids can be selected from the group consisting of: Lysine, Histidine, Arginine, Aspartic acid, Threonine, Serine, Glutamic acid, Proline, Glycine, Alanine, Cystine, Valine, Methionine, Isoleucine, Leucine, Norleucine, Tyrosine, and Phenylalanine.

The other additives can be selected from the group consisting of: Nitrogenous compounds, Enzymes, Proteins (Hemp protein), Glycoproteins, Sugars, Hydrocarbons, Simple alcohols, Aldehydes, Ketones, Simple acids, Simple esters, Lactones, Steroids, Pigments, and Vitamins.

In some embodiments, the cannabis ice product 22 may be flavored to enhance the flavor of the drink or may be flavorless such that the taste of the liquid 26 is unaltered as the cannabis ice product 22 melts into the liquid 26. The flavoring may be natural or artificial and can either be cannabis-flavored or can be a completely different flavor, e.g., a fruit flavor.

In some embodiments, the cannabis ice product 22 is preferably evenly spread throughout the cannabis ice product 22 such that it is released at a generally continuous concentration from the time the cannabis ice product 22 is inserted into the drink until it is fully melted.

In some embodiments, the cannabis ice product 22 may have a higher concentration in its middle than in its outer area such that as the cannabis ice product 22 melts and its surface area correspondingly falls, the release of the cannabis ice product 22 into the drink 20 is more constant from the time the cannabis product 22 is inserted into the drink until it fully melts. For example, in the embodiment of FIG. 6, the cannabis product 22 has a highest concentration in its center and the concentration decreases from the center to the outer surface. The decrease in concentration from the inside to the outside can be continuous or non-continuous. For example, a high concentration volume could be trapped within a low concentration area. In other embodiments, the cannabis ice product 22 may have a higher concentration of the cannabis product along its outer surface as compared to its middle so that the majority of the cannabis product contained in the cannabis ice product 22 is released quickly.

In an example embodiment, a single cannabis ice product 22 may contain one to fifty milligrams (1-50 mg) of THC. In some cases, cannabis ice products 22 having different amounts of THC can be produced. For example, one cannabis ice product 22 can have a dose of one to five milligrams (1-5 mg) of THC, another cannabis ice product 22 can have a dose of five to ten milligrams (5-10 mg) of THC, and yet another cannabis ice product 22 can have a dose of ten to fifty milligrams (10-50 mg) of THC. Cannabis ice product 22 having different quantities of THC than those listed here are also contemplated.

In an example embodiment, a single cannabis ice product 22 may contain ten to forty-five milligrams (10-45 mg) of CBD. In some cases cannabis ice product 22 having different amounts of CBD can be produced. For example, one cannabis ice product 22 can have a dose of ten to twenty milligrams (10-20 mg) of CBD, another cannabis ice product 22 can have a dose of twenty to thirty milligrams (20-30 mg) of CBD, and yet another cannabis ice product 22 can have a dose of thirty to forty-five milligrams (30-45 mg) of CBD. Cannabis ice product 22 having different quantities of CBD than those listed here are also contemplated.

Another aspect of the present disclosure is related to a method of making a cannabis ice product 22. The method begins with mixing a cannabis concentrate or product with a base constituent, such as liquid water, to form a cannabis liquid. The method proceeds with the step of pouring the cannabis liquid into a container, such as a mold or tray 28. In one embodiment, the interior of the tray 28 may be sized and shaped to directly form many cannabis ice products 22 in their final forms. In another embodiment, the interior of the tray 28 may be sized and shaped to make multiple cannabis ice products 22 simultaneously. In either case, the method continues with the step of freezing the cannabis liquid to form cannabis ice product 22. In other embodiments, the cannabis ice product 22 can be formed into a large sheet and cut to the final shapes. The cannabis liquid can be frozen in any suitable refrigeration manner.

In an example embodiment, the temperature of the cannabis ice product 22 is reduced to a temperature that is below (not at) the freezing point of the cannabis liquid so that it will not instantly begin to melt when exposed to temperatures above the freezing point in the drink 20 and to further slow the melting process once the cannabis ice product 22 is placed in the liquid 26. The cannabis ice product 22 may then be packaged and distributed.

In the drawings accompanying this patent application and the description above, various elements are identified using reference numbers to facilitate clarity and ease of reference. For instance, reference number 20 is used to denote a drink, while reference number 22 represents a cannabis ice product. In certain embodiments, similar components are designated with letters ‘a’, ‘b’, ‘c’, ‘d’, and ‘e’ to differentiate between variations or alternative embodiments of those components. For example, in one embodiment, the drink is identified as 20a, in another embodiment, as 20b, and in yet another embodiment, as 20c. The use of these reference numbers and letters assists in describing and distinguishing between different elements and variations thereof throughout the patent application.

The foregoing detailed description has been presented for purposes of illustration and description. It is not intended to be exhaustive or be limited to the precise form disclosed. Many modifications and variations are possible in light of the above description. The described embodiments were chosen to best explain the principles of the technology and its practical application to thereby enable others skilled in the art to best utilize the technology in various embodiments and with various modifications as are suited to the particular use contemplated. The scope of the technology is defined by the claims appended hereto.

What is claimed is:

1. A drink, comprising:
an ingestible liquid; and
a frozen cannabis product formed from a base constituent and at least one of tetrahydrocannabinol (THC) and cannabidiol (CBD) admixed with the base constitutent.

2. The drink as set forth in claim 1, wherein the base constituent is water.

3. The drink as set forth in claim 1, wherein the frozen cannabis product has a cube shape.

4. The drink as set forth in claim 1, wherein the frozen cannabis product has a spherical shape.

5. The drink as set forth in claim 1, wherein the at least one of THC and CBD is non-uniformly distributed across the frozen cannabis product.

6. The drink as set forth in claim 5, wherein a central area of the frozen cannabis product has a higher concentration of the at least one of THC and CBD than an outer area of the frozen cannabis product.

7. The drink as set forth in claim 1, wherein the frozen cannabis product includes ten to forty-five milligrams (10-45 mg) of CBD.

8. The drink as set forth in claim 1, wherein the frozen cannabis product includes one to fifty milligrams (1-50 mg) of THC.

9. A frozen cannabis product, comprising:
a base constituent admixed with at least one of tetrahydrocannabinol (THC) and cannabidiol (CBD) and frozen.

10. The frozen cannabis product as set forth in claim 9, wherein the base constituent is water.

11. The frozen cannabis product as set forth in claim 9, wherein the at least one of THC and CBD is non-uniformly distributed across the frozen cannabis product.

12. The frozen cannabis product as set forth in claim 11, wherein a central area of the frozen cannabis product has a higher concentration of the at least one of THC and CBD than an outer area of the frozen cannabis product.

13. The frozen cannabis product as set forth in claim 9, wherein the frozen cannabis product includes ten to forty-five milligrams (10-45 mg) of CBD.

14. The frozen cannabis product as set forth in claim 9, wherein the frozen cannabis product includes one to fifty milligrams (1-50 mg) of THC.

15. A method of making a cannabis product, comprising the steps of:
admixing a base liquid with at least one of tetrahydrocannabinol (THC) and cannabidiol (CBD) to form a cannabis mixture; and
freezing the cannabis mixture to form the frozen cannabis product.

16. The method as set forth in claim 15, wherein the frozen cannabis product includes ten to forty-five milligrams (10-45 mg) of CBD.

17. The method as set forth in claim 15, wherein the frozen cannabis product includes one to fifty milligrams (1-50 mg) of THC.

18. The method as set forth in claim 15, further including the step of inserting the frozen cannabis product into an ingestible liquid to form a drink.

19. The method as set forth in claim 15, wherein the base liquid is water.

20. The method as set forth in claim 15, wherein the frozen cannabis product has a spherical shape or a cube shape.
